(19) United States
(12) Patent Application Publication
(10) Pub. No.: US 2025/0065071 A1
(43) Pub. Date: Feb. 27, 2025

(54) FASTENING DEVICE FOR A PATIENT INTERFACE, PATIENT INTERFACE AND SYSTEM FOR VENTILATION AND/OR RESPIRATORY ASSISTANCE

(71) Applicant: Loewenstein Medical Technology S.A., Luxembourg (LU)

(72) Inventors: Martin BECHTEL, Winsen/Luhe (DE); Joachim Gardein, Icod de los Vinos (ES)

(21) Appl. No.: 18/801,906

(22) Filed: Aug. 13, 2024

(30) Foreign Application Priority Data
Aug. 24, 2023 (DE) 102023122679.7

(51) Int. Cl. A61M 16/06

(52) U.S. Cl. CPC A61M 16/0683

(57) ABSTRACT

A fastening device for a patient interface, comprising a substantially U-shaped support element having an apex and a first and second end section comprising a rigid and/or semirigid material, which encloses the back of the head of a patient when employed as intended, as well as a patient interface for use with a fastening device and a system for ventilation and/or respiratory assistance comprising a patient interface, a fastening device and a patient tube.

### CROSS-REFERENCE TO RELATED APPLICATIONS

The present application claims priority under 35 U.S.C. § 119 of German Patent Application No. 102023122679.7, filed Aug. 24, 2023, the entire disclosure of which is expressly incorporated by reference herein.

### BACKGROUND OF THE INVENTION

### 1. Field of the Invention

The present invention relates to a fastening device for a patient interface, comprising a substantially U-shaped support element having an apex and a first and second end section consisting of a rigid material, a semirigid material or mixtures thereof, which encloses the back of the head of a patient when employed as intended.

The present invention furthermore relates to a patient interface for use with a fastening device according to the invention, as well as to a system for ventilation and/or respiratory assistance comprising a patient interface, a patient tube and a fastening device according to the invention.

### 2. Discussion of Background Information

According to the present invention, patient interfaces are generally intended to mean respiratory masks consisting of a mask body and a mask cushion. These may be configured as a nasal mask, a full face mask, as a nasal pillow or nasal prong, as a mouthpiece, or as a hybrid mask comprising a mouthpiece and a nasal pillow. According to the present invention, patient interfaces are primarily used for treating sleep-related respiratory disorders, in particular obstructive sleep apnea syndrome, or impairments of the respiratory pump, for example chronic obstructive pulmonary disease (COPD), by using continuous positive airway pressure (CPAP) or noninvasive positive pressure ventilation. The patient interface is in this case connected via a patient tube to a ventilator or respiratory therapy apparatus. Patient interfaces which are small in their diameter, short in their height and low in their weight are then preferred. They have the advantage that the lever forces caused by the patient tube are small, the dead space volume is low and the patient interface is comfortable to wear.

Patient interfaces are fastened on the head of a patient via headgear. The latter is intended to hold the mask body securely on the head of a patient, only optimal positioning enabling the requisite holding without leaks occurring. Often, forehead supports or a plurality of wide belts are used for this purpose, which, although they allow better pressure distribution on the face of the patient, also lie very conspicuously in the field of view of a patient and very noticeably increase the overall weight. An increased number of application areas on the face of the patient due to wide belts may furthermore cause skin irritation, skin allergies and skin erosion, as well as chafing at sensitive places such as the ears.

There is an enormous demand for fastening devices for patient interfaces which obviate the need for forehead supports and wide belts or straps, which can thus be worn more comfortably and more conveniently by the patient and which do not significantly restrict the field of vision.

EP 3 085 405, the entire disclosure of which is incorporated by reference herein, discloses a headgear for fastening a respiratory mask having a first strap, which when employed as intended engages the posterior of the head of a patient and assumes a substantially circular shape, and having a pair of upper and lower straps which respectively extend over and under the ear of a patient and can receive a respiratory mask. The first strap comprises in this case encapsulated rigidizers as well as a first and second layer of fabric. The encapsulated rigidizers presented are capable of bending along their width, extension of the headgear as a whole in a longitudinal direction of the rigidizer being prevented.

EP 2 373 368 B1, the entire disclosure of which is incorporated by reference herein, discloses a headgear for use with a mask, having a first strap which, during use, assumes a substantially circular shape and receives the posterior of the head of a patient, a part of this first strap being inextensible, as well as an upper and a lower strap which respectively extend above and below the ear of a patient, the upper strap being more extensible than the lower strap.

The possibilities presented in the prior art have the aforementioned disadvantages of forehead supports or a plurality of wide belts on the face of the patient, such as a restricted field of view of the patient and, particularly when worn for a prolonged period of time, leaving behind pressure sores, skin irritations and chafing. Furthermore, the rigidizers disclosed therein are restricted in their extensibility, which does not allow adaptations to different head shapes and is uncomfortable when being worn, particularly in a lying position. Further, the rigidizers presented in the prior art are encapsulated in layers of fabric so that they are difficult to clean, which may lead to a buildup of bacteria that in turn increases the potential for skin irritation and allergy.

In view of the foregoing, it would be advantageous to have available fastening devices for patient interfaces which do not suffer from the disadvantages of the prior art and which allow the patient comfortable and convenient wearing of the patient interfaces, in particular even in a lying position. The fastening devices should offer secure holding of the patient interfaces as well as optimal positioning, without the field of view of the patient being excessively restricted and without the occurrence of skin irritations, skin allergies, skin erosion and chafing.

### SUMMARY OF THE INVENTION

The present invention provides a fastening device for a patient interface, comprising a substantially U-shaped support element having an apex, as well as a first and a second end section, configured and adapted to enclose the back of the head of a patient when employed as intended, wherein the U-shaped support element comprises a rigid material, a semirigid material or mixtures thereof.

The U-shaped support element according to the invention is configured in such a way here that it comprises no textile material, and can therefore be cleaned sterilely without great expense and has less wear due to abrasion. Furthermore, the material used is rigid enough to maintain its shape even when not being used by a patient. This allows the patient easier handling by the fastening device being simpler to position, or apply. This is often difficult with systems that comprise a plurality of textile belts or textile straps, since these systems become tangled and need to be laboriously straightened before use.

According to the present invention, U-shaped may as one alternative mean an arcuate U-shaped support element, in which the lower part in the section of the apex is configured as a semicircle. U-shaped support elements of this kind are preferred here in which such a semicircle has a radius of from about 4 cm to about 8 cm, in particular a radius of from about 5 cm to about 7 cm. Nevertheless, U-shaped elements of this kind are preferred in which the U-shape does not form an arc or semicircle but has a plateau in the section of the apex, that is to say the shape tends more toward a rectangle with rounded edges. Elements shaped in such a way have the advantage that they fit even better to the head shape of a patient.

Despite the use of rigid or semirigid material, the substantially U-shaped support element has a torsion in relation to a straight line that runs through the apex and centrally with respect to the first and second end section. Good adaptation to the respective situation of a patient, for example lying on the back or lying on the side, is thereby enabled.

Furthermore, it is likewise advantageous for the U-shaped support element according to the invention to have an extensibility in relation to a straight line that runs through the apex and centrally with respect to the first and second end section. This allows good adaptation to the respective head shape of a patient, particularly in respect of their head circumference, without leaving behind pressure sores as a result. The fastening device according to the invention may therefore in principle be used for many patients, without special different sizes of the U-shaped support element being required.

In one particularly preferred embodiment of the fastening device according to the invention, the apex of the U-shaped support element is adapted, during use on the patient, to bear centrally on their occipital bone. The first and the second end section of the U-shaped support element are each alternatively or in addition adapted, during use on the patient, to be positioned flat laterally on their head over the ears. In this configuration of the fastening device, the points of direct contact of the U-shaped support element when employed as intended thus lie on flat places of the head, such as the back of the head and laterally over the ears. The effect of this is that no skin irritations or wound areas occur due to friction or pressure.

U-shaped support elements in which the distance between the first and the second end section when not being employed is from about 8 cm to about 15 cm, in particular from about 10 cm to about 13 cm, are particularly highly suitable. Furthermore, such U-shaped support elements in which the distance between the apex and the first or second end section when not being employed is from about 14 cm to about 21 cm, in particular from about 16 cm to about 19 cm, are advantageous.

In particular embodiments, especially when the patient interface being used is configured as a full-face mask, it may alternatively be preferred for the U-shaped support element to be configured in particular predefined sizes, for example small, medium and large, in order to enable even better adaptation to different head sizes.

Furthermore, such fastening devices that further comprise at least one transition element, which connects the U-shaped support element to the patient interface, have proven particularly suitable. The at least one transition element is then preferably likewise formed from a rigid or semirigid material, or a mixture thereof. Fastening devices that comprise two transition elements, which respectively run on the left side and right side of the patient interface, are particularly highly suitable. The transition elements consisting of rigid or semirigid material impart the necessary stiffness or rigidity to the fastening device, in order to provide the requisite holding and the optimal positioning and to counteract the lever forces when attaching a patient tube, even without forehead supports or wide belts.

In order to restrict the field of view of a patient as little as possible, such fastening devices that further comprise at least one fabric band, which is used for connecting the U-shaped support element to the at least one transition element, have proven particularly suitable. In one alternative preferred configuration, the fastening device comprises two fabric bands. It is then in turn preferred for these two fabric bands to be used in embodiments with two transition elements.

The fabric bands are configured to be flexible and soft, and in the fastening device according to the invention they run over the face of the patient, specifically at places where excessive rigidity of the structure is not required and is even felt to be uncomfortable, such as the sensitive cheek region or in the vicinity of the eyes. These flexible soft fabric bands increase the wearing convenience for the patient enormously and do not restrict the field of view of the patient. Furthermore, the overall weight of the fastening device can be reduced by the use of lightweight fabric bands, which likewise contributes to a more comfortable wearing experience. The flexibility of the fabric bands furthermore enables better adaptation to the respective head shape of the patient, so that firm optimal seating of the patient interface can be guaranteed.

In addition, the one or two fabric bands of the fastening device according to the invention may comprise pad elements, which are preferentially fitted releasably on the inner side that faces toward the patient.

In order to connect the U-shaped support element to the at least one transition element by a first fabric band, or alternatively by a first and a second fabric band, it has proven advantageous for the U-shaped support element to have a reception element on the apex and reception elements on the two end sections.

Furthermore, it is then advantageous, alternatively or in addition, for the at least one transition element or alternatively the first and the second transition element to also comprise at least one reception element. Transition elements that comprise a first and a second reception element for receiving the fabric band or the fabric bands are particularly highly suitable.

In preferred configurations, the reception elements are configured in the form of slots, the slots having a length of from about 0.5 mm to about 6 mm, in particular from about 2 mm to about 4 mm, and a width of from about 10 mm to about 19 mm, in particular from about 13 mm to about 16 mm.

The connection of the U-shaped support element and the at least one transition element via releasable fabric bands offers the advantage that they can be separated easily from one another, which facilitates cleaning of the rigid or semirigid elements as well as the fabric bands. The components may thus be cleaned and disinfected separately, semirigid or rigid materials preferring different cleaning agents and concentrations than fabric bands, without material abrasion or material damage taking place. Furthermore, the releasable connection also allows changing or replacement of individual components if they suffer from wear.

Configurations of the fastening device according to the invention in which the reception elements are formed by press-in or feed-through elements are particularly suitable. These may for example be material recesses in the U-shaped support element and the transition elements, or protuberances for hooking, which are formed from the rigid or semirigid material itself. This offers the advantage of easy fastening by which lugs, press studs, hooks or the like, which could be uncomfortable for the patient when the fastening device is being worn because of possible friction, can be obviated. The feeding of the fabric bands through in the variant with material recesses is particularly preferably carried out in such a way that there is no ridge on the side facing toward the patient, at which the fabric band lies over the U-shaped support element, and the transition thus remains as flat as possible so that here again no pressure sores or friction occur.

In suitable configurations of the fastening device according to the invention, the feed-through elements in the U-shaped support element are configured in two pieces, in particular as two slot-shaped material recesses. It has proven advantageous in this case for the two slot-shaped recesses to run parallel to one another.

Alternatively or in addition, fastening devices in which the at least one transition element, in particular the first and the second transition element, are formed from a material which has a higher rigidity than the U-shaped support element are particularly highly suitable.

The transition elements of the fastening device according to the invention are seated close to, in particular on, the patient interface where the lever forces act more strongly after attachment of a patient tube, so that a certain stiffness of the system is necessary here.

Both in the case of the U-shaped support element and of the at least one transition element, in particular the first and second transition element, the rigid or semirigid material comprises polypropylene, polyethylene, polymethylpentene, polycarbonate, polystyrene, polyisobutylene, acrylonitrile butadiene styrene (ABS), polyvinylidene fluoride, polycaprolactone, polyethylene terephthalate, polybutylene terephthalate, ethylene-chlorotrifluoroethylene, polybenzimidazole, polyoxymethylene, ethylene-vinyl acetate copolymer, styrene-acrylonitrile copolymer, carbon nanotubes, cellulose acetate butyrate, thermoplastic elastomers and/or mixtures thereof.

The aforementioned materials can be cleaned better and more easily in comparison with a textile and have a higher robustness, particularly in relation to being abraded.

The rigid and/or semirigid material used for the U-shaped support element according to the invention as well as the at least one transition element, in particular the first and second transition element, is preferably configured with a thickness of from about 0.5 mm to about 4 mm, in particular from about 1.5 mm to about 2.5 mm. These material thicknesses allow the materials to be robust enough in order to be able to withstand the lever forces of a patient tube, but also flexible enough so that the fastening device according to the invention is comfortable and convenient for the patient.

In suitable configurations of the fastening device according to the invention, the at least one transition element, in particular the first and second transition element, further each comprise an attachment element for connection or reception of a patient interface. The attachment element may be configured here in the form of hooks, a screw-in mechanism, a hook-and-loop system or a latching mechanism (click system). A latching element is particularly preferred here. When required, the latter can be released as easily and simply as possible, for example when the patient experiences panic, wishes to drink something, or the like.

In preferred configurations of the fastening device according to the invention, the transition elements comprise gripping elements on the side facing away from the patient, so that the patient can grip the transition elements better in order to connect them to the patient interface. These gripping elements are preferably configured as ridges. Alternatively or in addition, the gripping elements comprise a structured surface, which is formed for example in the manner of ribs or roughened so that the patient can grasp them well with their fingers.

In order to be able to configure the transition elements as economically as possible in terms of material, substantially L-shaped transition elements have proven to be particularly highly suitable. Furthermore, this shape offers the best possible adaptation to the bone structure of the lower jaw of a patient and therefore feels comfortable for the patient to wear.

In preferred configurations, the side lengths of the L-shaped transition elements have a ratio of about 1.5:1, the reception elements preferably lying on the end sections of the shorter side. In preferred embodiments, the reception elements are configured as one-piece slot-shaped material recesses. These may run parallel or be offset by 90° here. For an optimal profile of the first and second fabric band, reception elements arranged parallel are preferred in nasal masks, while reception elements that are arranged offset substantially by about 90° have proven particularly highly suitable in face masks.

In suitable embodiments of the L-shaped transition element, the branches in the form of the longer and shorter side span an area xy, the L-shaped transition elements preferably not being configured to be flat in this plane. L-shaped transition elements in which one of the branches forms a ridge in the z plane are advantageous. The overall structure of the L-shaped transition elements can fit particularly well to the bone structure of the lower jaw of a patient by the resulting curved shape.

In preferred configurations, the transition elements are provided with at least one textile covering. Alternatively and/or in addition, the transition elements may comprise pad elements. The at least one textile covering may be wound around the transition elements and may be fastened on the at least one transition element via seams, hook-and-loop fasteners, zipper fasteners, press studs or lugs. Configurations in which a vasiform textile tube is slid over a transition element, and a fastener connection is not required, are particularly preferred. These fastener connections are liable to form ridges, which the patient feels to be uncomfortable during wearing and which may likewise lead to skin irritations and pressure sores. In any event, embodiments in which the textile covering is fastened on the transition elements without adhesives are preferred. Adhesives often release solvents in the course of time or decompose, which may lead to skin allergies, particularly for patients with sensitive skin.

In one suitable configuration, the fabric band according to the invention, in particular the first and second fabric band, are constructed in at least three layers with an inner layer facing toward the patient, at least one intermediate layer and an outer layer, which faces away from the patient. In one preferred configuration, the fabric bands comprise a laminated composite material, the at least three layers being connected to one another by means of flame lamination. Such a method offers the advantage that the layers are connected firmly to one another without the need to use adhesives or bonding agents, which in this case as well could lead to skin allergies or skin irritations.

Alternatively or in addition, such fabric bands which have rounded edges are preferred, which increases the wearing convenience for the patient and in particular does not lead to chafing at sensitive places, such as the ears.

For particularly comfortable wearing convenience, fabrics that have a smooth surface structure and are breathable are preferred. Ideally, these materials are furthermore straightforward to clean. Fabric bands whose inner or outer layer, or inner and outer layer, have a synthetic textile material or cellulosic chemical fibers are particularly suitable for this. These may comprise polyamides, regenerated polyamides, bio-polyamides, polyethylene terephthalate, regenerated polyethylene terephthalate, polyurethane, polypropylene, acrylonitrile, viscose, Lyocell, Rayon, Modal or mixtures thereof.

The textiles used in the fabric bands may be dyed here, which besides the appealing visual appearance may also be an aid to the patient when, for example, different fabric bands are used for different times of the day in combination with different patient interfaces (during the day, at night when sleeping), in which case the differently dyed fabric bands may possibly also be configured differently for this in their width or tensile strength. Alternatively or in addition, the differently dyed fabric bands may also have different application areas on the skin of a patient, so that the respective skin areas may be rested by changing the fabric bands.

During the production of such fabric bands, the already manufactured textile may in this case be dyed or alternatively the yarn used for production may be dyed. In preferred configurations of the fastening device according to the invention, the synthetic textile material of the fabric bands is dyed by means of dope dyeing. Color pigments are in this case already added to the plastic melt during the production of the filaments, the method being familiar to a person skilled in the art. Dope dyeing has proven to be particularly advantageous for the fastening devices according to the invention, in order to dye the fibers of the textile bands thoroughly and not just to obtain adhesion of the color pigments on the textile surface. The fabric bands used are often subjected to cleaning processes in order to be able to keep the fastening device sterile. Accordingly, with the method presented, a color fastness may be maintained even after multiple washing cycles. In principle, textiles dyed by dope dyeing have a significantly higher light fastness and longevity of the color pigments in comparison with subsequently dyed textiles. The fabric bands dyed in this way further offer the patient the advantage that less skin irritations and allergies occur. In the case of subsequently dyed textiles in which the color pigments adhere only on the surface, the likelihood that they will be separated from the fiber by sweat, friction and heat is significantly increased. Furthermore, the fabric bands dyed by dope dyeing of the fastening device according to the invention have a lower environmental impact and better biocompatibility.

In suitable configurations of the fastening device according to the invention, the first fabric band, in particular the first and the second fabric band, further comprise a clip in order to be able to be introduced into the reception element. The clip may be connected to the respective fabric band via a connecting element for this. All connecting elements familiar to a person skilled in the art, such as hooks, press studs, buttons, latching mechanisms, magnets, welding and the like may be envisioned for this. Configurations are particularly preferred in which the clip is stitched centrally onto the longer side of the respective fabric band, particularly in such a way that the clip and the fabric band are merely adjacent to one another and do not overlap, so that no ridge, which is felt to be uncomfortable by the patient when worn and leads to pressure sores, is formed on the fabric band.

Alternatively, configurations of the fastening device according to the invention are suitable in which the clip is connected to the fabric band so that they are not directly adjacent to one another but the clip can be moved relative to the fabric band, in particular so that size adaptation to different head sizes is possible. Such a connecting element may for example be formed by an elastic, a cord with a locking mechanism or particular configurations of the aforementioned connecting elements.

Alternatively or in addition, it can be provided that the clip, in particular the side of the clip that faces toward the patient, comprises a releasable pad element. This is preferred particularly in the variants of the movable clips when they bear on the neck of a patient.

Furthermore, configurations are preferred in which the fabric bands, in particular the fabric band that runs over the top of the head of a patient, have a marking element which runs centrally with respect to the first and second end of the fabric band. When employed as intended, that is to say when the fabric band is fed through the reception element of the U-shaped support element, this marking element lies centrally with respect to the end sections of the U-shaped support element and offers the patient an aid for placing the fastening device on the head in such a way that the marking element lies centrally on the back of the head. It is particularly advantageous in this case when the marking element likewise does not lead to any ridge of the fabric band that can lead to pressure sores. At the same time, however, the marking element should be perceptible not only by the sense of sight but also by the sense of touch, in order to be used as an aid for blind patients as well. In preferred configurations, the marking element is a flat seam which may be highlighted in color from the fabric band but can also be felt with the fingers. Alternatively, other marking elements which lead to a modified surface that is perceptible at this point may be envisioned, for example indentations, roughened areas on the side facing away from the patient, or the like.

Fastening devices according to the invention in which the first fabric band, in particular the first and the second fabric band, comprise a fixing element on their first and second band end are particularly highly suitable. These fixing elements are intended to connect the transition elements releasably to the fabric bands. In this case as well, all fixing elements familiar to a person skilled in the art may be envisioned, for example hooks, magnets, press studs, latching elements and the like. In one preferred configuration, such fixing elements which, besides their fixing functions, at the same time make it possible to allow optimal size adaptation to the head shape of the patient have proven particularly suitable. In one preferred configuration, the first and the second band end of the fabric bands therefore comprise hook-and-loop devices, which are configured in such a way that hook tapes are applied on the band ends of the fabric band, in particular stitched to the band ends. Preferably, the textile surface of the entire fabric bands is configured in such a way that it can act as a counterpart to the hook tape, so that the latter can be fixed at any desired place on the fabric band. Size adaptation to the respective head shape of the patient is therefore possible by simply relocating the band end that has the hook tape.

The present invention further provides a patient interface for use with a fastening device according to the invention.

In preferred configurations, the patient interface according to the invention comprises at least one engagement element, in particular a first and a second engagement element. This is used to receive the attachment elements of the first or the second transition element, preferably of the first and the second transition element. Preferably, the engagement elements are configured as a click system via a latching mechanism. Alternatively, hooks, screw-in mechanisms, hook-and-loop systems or similar connecting systems familiar to a person skilled in the art are possible. The preferred latching mechanism via a click system offers the advantage that it can be released as easily and rapidly as possible when required, for example when the patient experiences panic, wishes to drink something, or the like.

In suitable configurations of the patient interface according to the invention, it comprises a joining element in order to be able to receive a patient tube. Joining elements that are connected via a ball joint to the patient interfaces according to the invention are preferred here. In such embodiments, the patient interface therefore comprises an indentation, or material recess, which is suitable for receiving the ball joint.

The object of the invention is further achieved by a system for ventilation and/or respiratory assistance, comprising a patient interface according to the invention, a patient tube and a fastening device according to the invention, wherein the patient interface according to the invention and a ventilator and/or the respiratory therapy apparatus are in gas delivery communication with one another via the patient tube.

### BRIEF DESCRIPTION OF THE DRAWINGS

Further features and advantages of the invention may be found from the following description, in which exemplary embodiments of the invention are explained by way of example with the aid of schematic drawings, without thereby limiting the invention.

FIG. 1 shows a schematic structure of one embodiment variant of a U-shaped support element according to the invention;

FIG. 2 shows a schematic structure of one embodiment of a transition element according to the invention;

FIG. 3 shows a schematic structure of one embodiment of a fabric band according to the invention;

FIG. 4 shows a schematic structure of one embodiment of a fastening device according to the invention and

FIG. 5 shows a schematic structure of one embodiment of transition elements according to the invention connected to a patient interface according to the invention.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

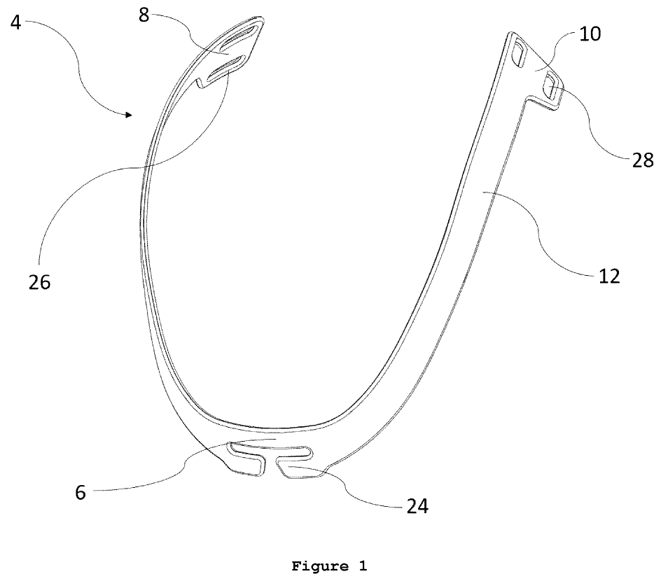

FIG. 1 shows a U-shaped support element 4 according to the invention, having an apex 6 as well as a first and a second end section 8 and 10. In the embodiment shown here, the U-shaped support element 4 has a plateau in the region of the apex 6. The U-shaped support element 4 consists in the variant shown of a rigid material 12, which maintains the U-shape even when it is not being used by a patient. In the embodiment shown, a torsion of the U-shaped support element 4 in relation to a straight line that runs through the apex 6 and centrally with respect to the first and second end section 8 and 10 is possible, that is to say the two arms of the U-shaped support element 4 can be twisted with respect to one another. Furthermore, the U-shaped support element 4 is extensible in relation to a straight line that runs through the apex 6 and centrally with respect to the first and second end section 8 and 10, that is to say the arms of the U-shaped support element 4 can be moved toward and away from one another. In the embodiment of the U-shaped support element 4 as shown, during use on the patient the apex 6 lies centrally on the occipital bone and the two end sections 8 and 10 are positioned laterally on the head over the ears. In the variant of the U-shaped support element 4 according to the invention as shown, it comprises a reception element 24 in the region of the apex 6, which is configured here in the form of a press-in element in which the rigid material 12 forms two protuberances into which a clip 52 of a fabric band 20 (not shown here) can be inserted so that a releasable connection is formed between the U-shaped support element 4 according to the invention and a fabric band 20. The embodiment of the U-shaped support element 4 according to the invention furthermore shows reception elements 26 and 28 on the two end sections 8 and 10. In the variant shown, these reception elements 26 and 28 are configured as two-piece feed-through elements so that a fabric band 20 can be fed through them (this is not shown). The feed-through elements are configured in this case as slot-like material recesses, which run parallel to one another here.

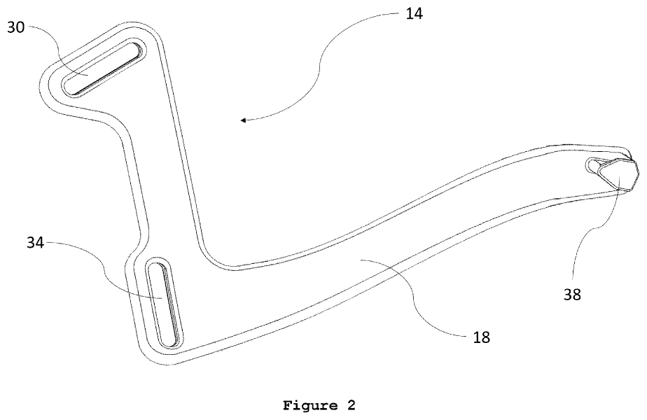

FIG. 2 shows a first transition element 14 according to the invention consisting of a rigid material 18, which is configured here as a left-side transition element 14 and is thus located on the left half of the face of the patient when worn. The transition element 14 shown is substantially configured in an L-shape here with a longer side and a shorter side, the ratio of the side lengths being 1.5:1 in this case.

In the variant shown, the transition element 14 comprises a first and a second reception element 30 and 34, through which a fabric band can be fed, on the two ends of the short side of the L-shape (not shown here). In the variant of the transition element 14 according to the invention as shown here, the reception elements 30 and 34 are configured as one-piece slot-like material recesses, the two reception elements 30 and 34 being offset at an angle of less than 90° with respect to one another here. In the embodiment of the first transition element 14 as shown, it has an attachment element 38 on the end of the long side of the L-shape for connection to a patient interface 2. In the variant shown here, this attachment element is configured as a latching element, the counterpart on the patient interface 2 having an associated reception device, the system thus forming a click system (not shown here).

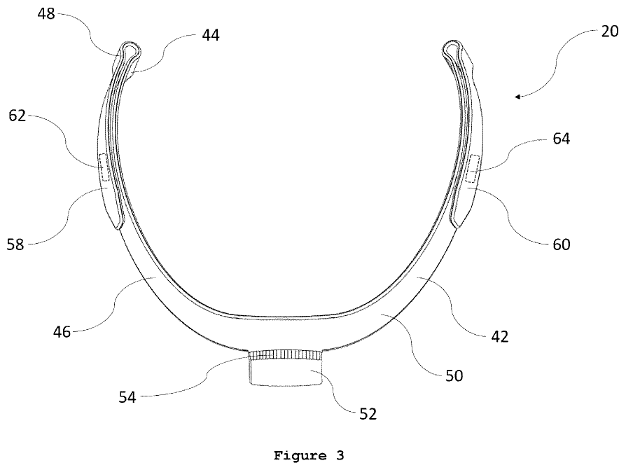

FIG. 3 shows a first fabric band 20 according to the invention. In the embodiment shown, it is produced from a laminated composite material 42 and comprises an inner layer 44 facing toward the patient, an outer layer 46 and an intermediate layer 48, the inner layer 44 and the outer layer 46 consisting of a synthetic textile material 50. The variant of the first fabric band 20 shown here comprises a clip 52 at the center of the fabric band 20, which is connected to the fabric band 20 via a connecting element 54, the connecting element 54 being configured here as a seam. In order to generate a transition that is as flat as possible, the fabric band 20 and the clip 52 were in this case stitched in such a way that they are only adjacent to one another but do not overlap. The clip 52 shown is configured in order to be introduced releasably into the reception element 24 of the U-shaped support element 4 according to FIG. 1. The fabric band 20 shown has a first and a second band end 58 and 60. In the variant shown, the two band ends 58 and 60 each have a fixing element 62 and 64, which is applied here on the outer layer 46 that faces away from the patient, in the form of a hook tape. The latter is configured here in the variant shown in such a way that it can be connected to the entire outer layer 46 after being folded over. The band ends 58 and 60 are suitable for being fed through the reception elements 26 and 28 of the U-shaped support element 4 of FIG. 1.

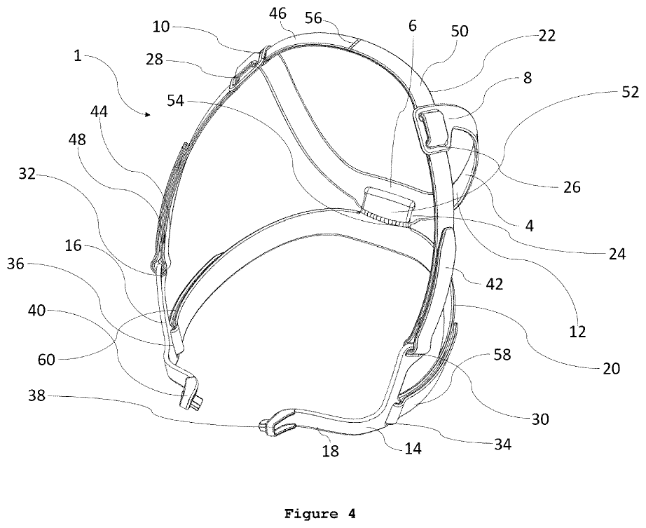

FIG. 4 shows a fastening device 1 according to the invention for a patient interface 2 (not shown here). In the embodiment shown here, the U-shaped support element 4 according to FIG. 1 is connected to a first transition element 14 according to FIG. 2 and a further second transition element 16, which then forms the right-side transition element and thus lies on the right half of the face of a patient, via a first fabric band 20 according to FIG. 3 and a further fabric band 22. In the variant shown here, the first fabric band 20 is introduced via the clip 52 into the reception element 24 on the apex 6 of the U-shaped support element 4, and furthermore via the band ends 58 and 60 into the second reception elements 34 and 36 of the transition elements 14 and 16. The second fabric band 22 is initially fed via its band ends 58 and 60 through the reception elements 26 and 28, which are configured here as feed-through elements, and furthermore through the first reception elements 30 and 32 of the transition elements 14 and 16. The feeding through the reception elements 26 and 28, which are configured here as two-piece material recesses, is then carried out in such a way that a ridge is formed on the side facing away from the patient by the second fabric band 22 bearing partially on the U-shaped support element 4, and is not formed on the inner side facing toward the patient. The second fabric band 22 is placed here between the first and second end section 8 and 10 of the U-shaped support element 4 so that the marking element 56 is positioned centrally with respect to these end sections 8 and 10. When the fastening device 1 according to the invention is being used as intended, the marking element 56 helps the patient to be able to place the fastening device 1 centrally on the back of the head. In the variant of the fastening device 1 according to the invention as shown, the branches in the form of the longer and shorter side of the L-shaped transition elements 14 and 16 respectively span an area xy. The L-shaped transition elements 14 and 16 are not configured to be flat in the area xy in the embodiment shown, but form a ridge in the z plane. The two L-shaped transition elements 14 and 16 are therefore aligned convergently with one another in order to be able to receive a patient interface 2 (not shown). In the variant shown, the L-shaped transition elements 14 and 16 further have arcuate gripping structures on the side facing away from the patient, which are intended to make it easier for the patient to grip the transition elements 14 and 16 when fastening to a patient interface 2 (not shown).

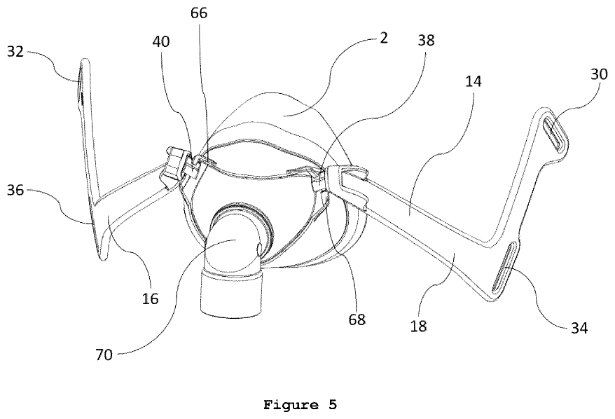

FIG. 5 shows the transition elements 14 and 16 already shown in FIG. 4, these being connected here to a patient interface 2 via their attachment elements 38 and 40. In the variant shown here, the patient interface 2 comprises two engagement elements 66 and 68, which receive the attachment elements 38 and 40 of the transition elements 14 and 16. This is controlled in the embodiment shown via a latching mechanism. The patient interface 2 shown furthermore comprises a joining element 70 for receiving a patient tube 102 (not shown here). In the embodiment shown here, the joining element 70 comprises a ball joint and the patient interface 2 comprises a spherical material recess for receiving this ball joint (not shown here).

The features of the invention which are disclosed in the description above, in the claims and in the drawings may be essential either individually or in any desired combination for implementation of the invention in its various embodiments.

### LIST OF REFERENCE SIGNS

1 fastening device
2 patient interface
4 U-shaped support element
6 apex of the U-shaped support element
8 first end section of the U-shaped support element
10 second end section of the U-shaped support element
12 rigid and/or semirigid material of the U-shaped support element
14 first transition element
16 second transition element
18 rigid and/or semirigid material of the transition elements
20 first fabric band
22 second fabric band
24 reception element on the apex
26 reception element on the first end section
28 reception element on the second end section
30 first reception element of the first transition element
32 first reception element of the second transition element
34 second reception element of the first transition element
36 second reception element of the second transition element
38 attachment element of the first transition element
40 attachment element of the second transition element
42 laminated composite material
44 inner layer facing toward the patient
46 outer layer facing away from the patient
48 intermediate layer
50 synthetic textile material
52 clip
54 connecting element
56 marking element
58 first band end
60 second band end
62 fixing element on the first band end
64 fixing element on the second band end
66 first engagement element
68 second engagement element
70 joining element
102 patient tube

What is claimed is:

1. A fastening device for a patient interface, wherein the device comprises a substantially U-shaped support element having an apex and comprising a rigid and/or semirigid material, as well as a first end section and a second end section, configured and adapted to enclose a back of a head of a patient when employed.

2. The fastening device of claim 1, wherein the apex is configured and adapted, during use on the patient, to bear centrally on their occipital bone and the first end section and the second end section are each configured and adapted, during use on the patient, to be positioned flat laterally on their head over ears.

3. The fastening device of claim 1, wherein the device further comprises at least one transition element, configured and adapted to connect the U-shaped support element to the patient interface, the at least one transition element comprising a rigid and/or semirigid material.

4. The fastening device of claim 3, wherein the device further comprises at least one fabric band which is configured and adapted to connect the U-shaped support element to the at least one transition element.

5. The fastening device of claim 4, wherein the U-shaped support element comprises a reception element on the apex and a reception element on each of the two end sections, configured and adapted to receive the at least one fabric band.

6. The fastening device of claim 4, wherein the at least one transition element comprises at least one reception element, configured and adapted to receive the at least one fabric band.

7. The fastening device of claim 3, wherein a first transition element and/or a second transition element are formed from a material which has a higher rigidity than the U-shaped support element.

8. The fastening device of claim 3, wherein a first transition element and/or a second transition element further comprises an attachment element for connection to a patient interface.

9. The fastening device of claim 3, wherein a first transition element and/or a second transition element are substantially configured in an L-shape.

10. The fastening device of claim 4, wherein a first fabric band and/or a second fabric band comprises a laminated composite material having an inner layer facing toward a patient, an outer layer and at least one intermediate layer.

11. The fastening device of claim 10, wherein the inner layer and/or the outer layer comprises a synthetic textile material and/or comprises cellulosic chemical fibers, the synthetic textile material being dyed by dope dyeing.

12. The fastening device of claim 4, wherein a first fabric band and/or a second fabric band comprise a clip which is configured and adapted to be introduced into a reception element, and/or wherein the first fabric band and/or the second fabric band and the clip are connected to one another via a connecting element.

13. The fastening device of claim 4, wherein a first fabric band and/or a second fabric band comprise a marking element running centrally with respect to a first band end and a second band end, the marking element being configured and adapted to be seated centrally with respect to two end sections when the first fabric band and/or the second fabric band is fed through reception elements of the U-shaped support element.

14. The fastening device of claim 4, wherein the device further comprises a fixing element on a first band end and on a second band end of a first fabric band and/or a second fabric band, configured and adapted to receive transition elements for connection to a patient interface.

15. The fastening device of claim 14, wherein the fixing elements are configured and adapted to receive the transition elements so that size adaptation to a head shape of the patient is simultaneously enabled.

16. A patient interface suitable for use with the fastening device of claim 1.

17. The patient interface of claim 16, wherein the interface comprises at least one engagement element which is configured and adapted to receive attachment elements of a first transition element and/or a second transition element of the fastening device.

18. The patient interface of claim 16, wherein the interface further comprises a joining element which is configured and adapted to receive a patient tube, the joining element being configured as a ball joint.

19. A system for ventilation and/or respiratory assistance, wherein the system comprises a patient interface, a fastening device and a patient tube, the patient interface being in gas delivery communication via the patient tube with a ventilator and/or a respiratory therapy apparatus, the fastening device being configured as set forth in claim 1.

20. A system for ventilation and/or respiratory assistance, wherein the system comprises a patient interface, a fastening device and a patient tube, the patient interface being in gas delivery communication via the patient tube with a ventilator and/or a respiratory therapy apparatus and being configured as set forth in claim 16.
